(19) United States
(12) Patent Application Publication
(10) Pub. No.: US 2019/0025240 A1
(43) Pub. Date: Jan. 24, 2019

(54) Electrode Integration Into Organs On Chip Devices

(71) Applicant: President and Fellows of Harvard College, Cambridge, MA (US)

(72) Inventors: Olivier Henry, Brookline, MA (US); Andries van der Meer, Enschede (NL); Donald E. Ingber, Boston, MA (US)

(21) Appl. No.: 16/062,923

(22) PCT Filed: Dec. 16, 2016
(86) PCT No.: PCT/US16/67294

§ 371 Date: Jun. 15, 2018

Related U.S. Application Data
(60) Provisional application No. 62/268,454, filed on Dec. 16, 2015.
(60) Provisional application No. 62/297,659, filed on Feb. 19, 2016.

(51) Int. Cl. G01N 27/30; C12M 1/34; B01L 3/00

(52) U.S. Cl. CPC G01N 27/305; C12M 41/46; B81C 3/00; B01L 3/502707; B01L 3/502715

(57) ABSTRACT

A method of fabricating electrodes includes forming a first metallic film layer on an upper surface of a first material substrate, and attaching a first polymeric layer to the upper surface of the first material substrate to form a first opened microchannel. The method further includes forming a second metallic film layer on a portion of a lower surface of a second material substrate, and attaching a second polymeric layer to the lower surface of the second material substrate to form a second opened microchannel. The method also includes attaching the first opened microchannel to a bottom side of the membrane and the second opened microchannel to the top side of the membrane. The first metallic film layer and the second metallic film layer each constitute transparent electrodes and are positioned with the membrane therebetween.

### CROSS-REFERENCE TO RELATED APPLICATIONS

This application claims priority to and benefit of U.S. Provisional Patent Application Ser. No. 62/268,454, filed on Dec. 16, 2015, and U.S. Provisional Patent Application Ser. No. 62/297,659, filed on Feb. 19, 2016, each of which is hereby incorporated by reference herein in its entirety.

### GOVERNMENT SUPPORT

The invention was made with Government Support under Contract No. DE-FG02-02ER63445 awarded by the U.S. Department of Energy. The government has certain rights in the invention.

### FIELD OF THE INVENTION

The present invention relates generally to electrode integration into organ-on-chip devices, and, more particularly, to fabrication of electrodes for microchannel devices.

### BACKGROUND OF THE INVENTION

A number of in situ analytical technique commonly used to assess cell culture growth, viability or death are not transferable to the microfluidics design used in the preparation of organs on chip (“OOC”). For example, trans epithelial electrical resistance (“TEER”) measures the growth and packing of cell. In standard cell culture in transwells, in which cells are cultured on a thin permeable membrane with media present above and below the membrane, macro electrodes are easily inserted above and below the membrane. The electrical resistance measured between the two electrodes is a good indication of cell packing and the existence or absence of tight junctions between cells. This approach, however, is not compatible with the OCC design.

Attempts have been made to take TEER measurements using metallic inlet/outlet ports as electrodes, as well as inserting silver/silver chloride reference electrodes in the ports. Although mathematical models were developed, the results remain difficult to interpret.

Other reports refer to the fabrication of gold electrodes onto a glass substrate, which is, then, integrated into a polydimethoxysiloxane (“PDMS”) device. Yet other reports refer to insertion of wire electrodes in PDMS devices. However, no commercial systems exist that have electrodes integrated with microfluidics for TEER compatible with the OOC approach.

Lastly, Applied Biophysics Inc. offers a number of planar electrode arrays in a single channel (http://www.biophysics.com). For example, ACEA Biosciences Inc. proposes electrodes integrated onto membranes for cell cultures mounted into microtitre plates (xCELLigence, ACEA Biosciences Inc./Roche, http://www.aceabio.com/). However, this also fails to provide a suitable option for the OOC approach.

### SUMMARY OF THE INVENTION

According to one embodiment of the present invention, a method is directed to fabricating electrodes for a microchannel device having a membrane, and includes forming a first electrically conductive film layer on a portion of an upper surface of a first material substrate. The method also includes attaching a first polymeric layer defining the dimensions of the microfluidic channel to the upper surface of the first material substrate to form a first opened microchannel containing the first electrically conductive film layer, the first electrically conductive film layer extending across the first opened microchannel. The method further includes forming a second electrically conductive film layer on a portion of a lower surface of a second material substrate, and attaching a second polymeric layer defining the dimensions of the microfluidic channel to the lower surface of the second material substrate to form a second opened microchannel, the second electrically conductive film layer extending across the second opened microchannel. The method further includes attaching the first opened microchannel containing the first electrically conductive film layer to a bottom side of the membrane and the second opened microchannel containing the second electrically conductive film layer to the top side of the membrane, the first electrically conductive film layer and the second electrically conductive film layer each constituting electrodes and being positioned with the membrane therebetween.

According to one aspect of the method described above, the first electrically conductive film layer, the first material substrate, and the first polymeric layer defining the dimensions of the microfluidic channel form a first microchannel assembly, the second electrically conductive film, the second material substrate, and the second polymeric layer defining the dimensions of the microfluidic channel form a second microchannel assembly, the first microchannel assembly and the second microchannel assembly being symmetrical.

According to yet another aspect of the method described above, the method further includes integrating a plurality of electrical contacts into the first material substrate and the second material substrate and/or the membrane, each of the plurality of electrical contacts being electrically coupled with a respective end of the first electrically conductive film layer and the second electrically conductive film layer.

According to yet another aspect of the method described above, the method further includes integrating a connection to the plurality of electrical contacts for enabling connecting the electrodes to external electronics and instrumentation.

According to yet another aspect of the method described above, the material substrate is a polymer, including polycarbonate, styrene-ethylene/butylene-styrene (“SEBS”), polydimethylsiloxane, polyurethane, polyester, cyclic olefin copolymer (“COC”), cyclic olefin polymer (“COP”), SU-8, polymethylmethacrylate (“PMMA”), polyvinyl chloride (“PVC”), polystyrene (“PS”), and/or polyethylene terephthalate (“PET”).

According to yet another aspect of the method described above, the material substrate is glass, silicon, and/or silicon nitride.

According to yet another aspect of the method described above, the membrane is a polymer, including polycarbonate, SEBS, polydimethylsiloxane, polyurethane, polyester, COC, COP, silicon nitride, SU-8, PMMA, PVC, PS, and/or PET.

According to yet another aspect of the method described above, the membrane is glass, silicon, and/or silicon nitride.

According to yet another aspect of the method described above, the membrane is a natural polymer.

According to yet another aspect of the method described above, the membrane is a biodegradable polymer.

According to yet another aspect of the method described above, at least one of the attaching steps includes a curing process at approximately 60° Celsius.

According to yet another aspect of the method described above, at least one of the first electrically conductive film layer, the first material substrate, the first polymeric layer, the second electrically conductive film layer, the second material substrate, and the second polymeric layer includes at least one material selected from a group consisting of a flexible material and a stretchable material.

According to yet another aspect of the method described above, the electrically conductive film covers entirely or partially the microchannel.

According to yet another aspect of the method described above, the electrodes are placed perpendicular or parallel to the microchannel.

According to yet another aspect of the method described above, at least one of the first electrically conductive film and the second electrically conductive film has a thickness in the range of approximately 10-30 nanometers.

According to yet another aspect of the method described above, at least one of the first electrically conductive film and the second electrically conductive film consists of a plurality of layers including one or more titanium layers and at least one gold layer.

According to yet another aspect of the method described above, the plurality of layers includes a first titanium layer having a thickness of about 3 nanometers, a second gold layer having a thickness of about 25 nanometers, and a third titanium layer having a thickness of about 1 nanometers.

According to yet another aspect of the method described above, the electrodes include a material selected from a group consisting of a metal, a semi-conductor, an oxide, a carbon, and a polymer.

According to yet another aspect of the method described above, the metal includes a material selected from a group consisting of gold, platinum, silver, and silver chloride.

According to yet another aspect of the method described above, the semi-conductor is doped silicon.

According to yet another aspect of the method described above, the oxide includes a material selected from a group consisting of indium tin oxide, titanium dioxide, and graphene oxide.

According to yet another aspect of the method described above, the carbon includes a material selected from a group consisting of graphite, fullerenes, and graphene.

According to yet another aspect of the method described above, the polymer includes a material selected from a group of conductive polymers consisting of doped polyaniline, undopped polyaniline, polypyrrole, and polythiophene.

According to yet another aspect of the method described above, the polymer is conductive or semi-conductive via addition of conducting or semi-conducting species.

According to yet another aspect of the method described above, the conducting or semi-conducting species are selected from a group consisting of nanoparticles and carboneous elements.

According to yet another aspect of the method described above, the carboneous elements are selected from a group consisting of carbon black, graphite, carbon nanotubes, fullerenes, graphene, and a combination thereof.

According to yet another aspect of the method described above, the electrodes are coated with a conductive or insulating layer.

According to yet another aspect of the method described above, the conductive or insulating layer is selected from a group consisting of one or more polymers, organic mono-layers, organic polylayers, and oxides.

According to yet another aspect of the method described above, the polymers are selected from a group consisting of epoxy-based negative photoresist SU-8, and silicon nitride.

According to yet another aspect of the method described above, the organic mono-layers or organic polylayers include a self-assembled monolayer of thiolated compounds or silane.

According to yet another aspect of the method described above, at least one of the electrodes is transparent to light.

According to yet another aspect of the method described above, at least one of the electrodes has a thickness such that it is transparent to light.

According to yet another aspect of the method described above, at least one of the electrodes has a thickness in the range of approximately 1 nanometers to 100 micrometers, and preferably in the range of approximately 10-50 nanometers.

According to yet another aspect of the method described above, at least one of the electrodes is flexible. By way of example, flexible materials for the electrodes include polycarbonate, PET, and KAPTON® rubber having a flexular modulus typically between 1 and 6 gigapascals (“GPa”).

According to yet another aspect of the method described above, at least one of the electrodes is stretchable. By way of example, stretchable materials for the electrodes include PDMS, SEBS, or rubber having a Young's modulus less than 1 GPa.

According to yet another aspect of the method described above, one or more of the first electrically conductive film layer and the second electrically conductive film layer are disposed on the membrane.

According to yet another aspect of the method described above, one or more of the first electrically conductive film layer and the second electrically conductive film layer are disposed on the membrane by any suitable method, including, but no limited to, deposition, vapor deposition, precipitation, spraying, ablating, masking, etching, printing, and/or contact printing.

According to yet another aspect of the method described above, the method further includes forming a third electrically conductive film layer on a portion of the bottom side of the membrane or the top side of the membrane, the third electrically conductive film layer constituting another electrode.

According to yet another aspect of the method described above, the method further includes forming a fourth electrically conductive film layer on a portion of the other of the bottom side of the membrane or the top side of the membrane, the fourth electrically conductive film layer constituting another electrode.

According to yet another aspect of the method described above, at least one of the first electrically conductive film layer and the second electrically conductive film layer is a metallic film layer.

According to another embodiment of the present invention, a device contains electrodes and includes a body having a first microchannel and a second microchannel. The device further includes a membrane located at an interface region between the first microchannel and the second microchannel, the membrane including a first side facing toward the first microchannel and a second side facing toward the second microchannel, the first side having cells adhered thereto. The device further includes a first electrode positioned on a first side of the membrane and a second electrode positioned on a second side of the membrane.

According to one aspect of the device described above, the first electrode is symmetrically integrated with respect to the second electrode.

According to another aspect of the device described above, the device further includes electrical contacts directly integrated in one or more of the body and the membrane such that each is electrically coupled with a respective end of the first and second electrodes.

According to yet another aspect of the device described above, at least one of the body, the membrane, and the electrodes includes at least one material selected from a group consisting of a flexible material and a stretchable material.

According to yet another aspect of the device described above, at least one of the electrodes has a thickness in the range of approximately 10-30 nanometers.

According to yet another aspect of the device described above, at least one of the electrodes has a plurality of layers including one or more titanium layers and at least one gold layer.

According to yet another aspect of the device described above, at least one of the first electrode and the second electrode is transparent to light.

According to yet another aspect of the device described above, at least one of the first electrode and the second electrode has a thickness such that it is transparent to light.

According to yet another aspect of the device described above, at least one of the first electrode and the second electrode has a thickness in the range of approximately 1 nanometers to 100 micrometers, and preferably in the range of approximately 10-50 nanometers.

According to yet another aspect of the device described above, at least one of the first electrode and the second electrode is flexible.

According to yet another aspect of the device described above, at least one of the first electrode and the second electrode is stretchable.

According to yet another aspect of the device described above, one or more metallic film layers are disposed on the membrane.

According to yet another embodiment of the present invention, a method is directed to measuring electrical characteristics across a membrane, and includes (a) providing a microfluidic device having i) a first microfluidic channel, ii) a second microfluidic channel, iii) a semipermeable membrane disposed between the first microfluidic channel and the second microfluidic channel, the semipermeable membrane comprising first and second surfaces, iv) a first culture of cells on the first surface of the semipermeable membrane, and a second culture of cells on the second surface of the semipermeable membrane, and v) a first electrode in fluid communication with the first microfluidic channel and a second electrode in fluid communication with the second microfluidic channel, wherein the first and second electrodes are transparent. The method further includes (b) measuring electrical characteristics across the semipermeable membrane using the first and second electrodes.

According to one aspect of the method described above, the method further includes (c) observing the cells through either the first or second transparent electrodes.

According to another aspect of the method described above, the first and second electrodes include gold having a thickness such that it is transparent to light.

According to yet another aspect of the method described above, the thickness of the gold is 25 nanometers or less.

According to yet another aspect of the method described above, the first culture of cells includes epithelial cells and the measuring includes measuring transepithelial electric resistance (TEER).

According to yet another embodiment of the present invention, a method is directed to measuring electrical characteristics across a membrane, and includes a) providing a microfluidic device including i) a first microfluidic channel, ii) a second microfluidic channel, iii) a semipermeable membrane disposed between the first microfluidic channel and the second microfluidic channel, iv) a first culture of cells in the first microfluidic channel, and v) a first electrode in fluid communication with the first microfluidic channel and a second electrode in fluid communication with the second microfluidic channel, wherein the first and second electrodes are transparent. The method further includes b) measuring electrical characteristics across the membrane using the first and second electrodes.

According to one aspect of the method described above, the membrane includes first and second surfaces, the first culture of cells being on the first surface of the semipermeable membrane.

According to another aspect of the method described above, the microfluidic device further includes a second culture of cells on the second surface of the semipermeable membrane.

According to yet another aspect of the method described above, the method further includes the step of c) observing the cells through either the first or second transparent electrodes.

According to yet another aspect of the method described above, the first and second electrodes include gold having a thickness such that it is transparent to light.

According to yet another aspect of the method described above, the thickness of the gold is 25 nanometers or less.

According to yet another aspect of the method described above, the first culture of cells include at least one of epithelial cells and endothelial cells.

According to yet another aspect of the method described above, the measuring includes measuring one or more of Transepithelial Electric Resistance (TEER), short circuit current, cell capacitance, electric stimuli to cell cultures, localized degradation of cell layer, cell proliferation, cell migration across substrate, cell migration across membrane, physical stress applied to the microfluidic device, mechanical stress applied to the microfluidic device, flow rate of a fluid flowing in the microfluidic device, formation of bubbles, and functionalize of electrodes.

According to yet another embodiment of the present invention, a method is directed to measuring electrical characteristics across a membrane, the method including a) providing a microfluidic device including i) a first microfluidic channel, ii) a second microfluidic channel, iii) a semipermeable membrane disposed between the first microfluidic channel and the second microfluidic channel, iv) a first culture of cells in the first microfluidic channel, and v) electrodes in fluid communication with the first microfluidic channel. The method further includes b) measuring electrical characteristics across the membrane by impedance spectroscopy.

According to one aspect of the method described above, the membrane includes first and second surfaces, the first culture of cells being on the first surface of the semipermeable membrane.

According to another aspect of the method described above, the microfluidic device further includes a second culture of cells on the second surface of the semipermeable membrane.

According to yet another embodiment of the present invention, a device includes a membrane positioned between a top electrode and a bottom electrode, the top and bottom electrodes being connected to a detachable interface.

According to one aspect of the device described above, the device is a microfluidic device and the membrane is positioned between first and second microchannels.

According to another aspect of the device described above, the microfluidic device includes cells in the first or second microchannels, or both.

According to yet another embodiment of the present invention, a method is directed to connecting external test instruments to electrodes integrated into a microfluidic device, and includes providing a microfluidic device including a first microfluidic channel, a second microfluidic channel, a semipermeable membrane disposed between the first microfluidic channel and the second microfluidic channel, a first culture of cells in the first microfluidic channel, and electrodes in fluid communication with the first microfluidic channel. The method further includes connecting the electrodes to one or more external instruments.

According to yet another embodiment of the present invention, a device containing electrodes includes a body having a first microchannel and a second microchannel, and a membrane located at an interface region between the first microchannel and the second microchannel. The membrane includes a first side facing toward the first microchannel and a second side facing toward the second microchannel, the first side having cells adhered thereto. The device further includes an electrode positioned on one side of the membrane.

According to one aspect of the device described above, the device further includes another electrode positioned on another side of the membrane.

According to another aspect of the device described above, the device further includes one or more additional electrodes on at least one of the first microchannel and the second microchannel.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1A illustrates a first step of a fabrication process with polycarbonate base substrates.

FIG. 1B illustrates a second step of the fabrication process of FIG. 1A.

FIG. 1C illustrates a third step of the fabrication process of FIG. 1A.

FIG. 1D illustrates a fourth step of the fabrication process of FIG. 1A.

FIG. 1E illustrates a fifth step of the fabrication process of FIG. 1A.

FIG. 2 is a perspective view illustrating a device fabricated with the fabrication process of FIG. 1.

FIG. 3 is a chart illustrating electrode electrochemical impedance spectroscopy measured at different time points during the culture of Caco-2 cells.

FIG. 4A is a low magnification phase contrast image representative of day one of Caco-2 cells cultured in a prototype TEER device.

FIG. 4B is a low magnification phase contrast image representative of day five of the Caco-2 cells cultured in the prototype TEER device of FIG. 4B.

FIG. 5 is a chart illustrating a time course experiment of Air-Liquid Interface (“ALP”) formation in a small-airway chip with integrated electrodes.

FIG. 6 is a diagram illustrating a method of measuring electrical characteristics across a membrane.

FIG. 7A is a plot illustrating data for Caco2 cells grown static conditions.

FIG. 7B is a plot illustrating data for Caco2 cells under flow conditions.

FIG. 7C is a fluorescent confocal micrograph showing vili-like structures formed under flow conditions.

FIG. 7D is a circuit diagram for extracted values from the data presented in FIGS. 7A and 7B.

FIG. 7E is a plot illustrating evolution of TEER values during a culture time for the Caco2 cells of FIGS. 7A and 7B.

FIG. 7F is a plot illustrating evolution of Capacitance values during the culture time for the Caco2 cells of FIGS. 7A and 7B.

FIG. 8A is an exploded view of a TEER chip, according to an alternative embodiment.

FIG. 8B is an assembled view of the TEER chip of FIG. 8A.

FIG. 9 is a perspective view illustrating an electric connection between OOC integrated electrodes and external instrumentation.

FIG. 10 is a perspective view illustrating an OOC device with electrodes for measurement of parameters to which physical stress is applied.

FIG. 11 is a plot illustrating stress measurements.

FIG. 12A is a perspective view illustrating a sealable interface.

FIG. 12B is a perspective view illustrating the sealable interface of FIG. 12A with a top compression plate.

FIG. 12C is a perspective view illustrating the sealable interface of FIG. 12B with a fluidic inlet and outlet connections.

FIG. 13 is a perspective view illustrating the sealable interface of FIG. 12C with the fluidic inlet and outlet connections, and with electrical inlet and outlet connections.

FIG. 14 is an enlarged perspective view of some of the fluidic inlet and outlet connetions and the electrical inlet and outlet connections.

FIG. 15 is a perspective view of a printed circuit board.

FIG. 16 is a top view of the printed circuit board of FIG. 15.

FIG. 17 is a perspective view of an automated digital microfluidic platform.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

While this invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated. For purposes of the present detailed description, the singular includes the plural and vice versa (unless specifically disclaimed); the words “and” and “or” shall be both conjunctive and disjunctive; the word “all” means “any and all”; the word “any” means “any and all”; and the word “including” means “including without limitation.”

### Definitions

The term “microfluidic” as used herein relates to components where a moving fluid is constrained in or directed through one or more channels in which one or more dimensions are 1 millimeter (“mm”) or smaller (microscale). Microfluidic channels may be larger than microscale in one or more directions, though the channel(s) will be on the microscale in at least one direction. In some instances, the geometry of a microfluidic channel is configured to control the fluid flow rate through the channel (e.g. increase channel height to reduce shear). Microfluidic channels are formed of various geometries to facilitate a wide range of flow rates through the channels.

“Channels” are pathways (whether straight, curved, single, multiple, in a network, etc.) through a medium (e.g., silicon) that allow for movement of liquids and gasses. Channels, thus, connect other components, i.e., keep components “in communication” and more particularly, “in fluidic communication,” and still more particularly, “in liquid communication.” Such components include, but are not limited to, liquid-intake ports and gas vents. Microchannels are channels with dimensions less than 1 mm and greater than 1 micron.

As used herein, the phrases “connected to,” “coupled to,” “in contact with,” and “in communication with” refer to any form of interaction between two or more entities, including mechanical, electrical, magnetic, electromagnetic, fluidic, and thermal interaction. For example, in one embodiment, channels in a microfluidic device are in fluidic communication with cells and (optionally) a fluid source, such as a fluid reservoir. Two components are coupled to each other even if they are not in direct contact with each other. For example, two components are coupled to each other through an intermediate component (e.g., tubing or other conduit).

Full Integration of Electrodes in OOC

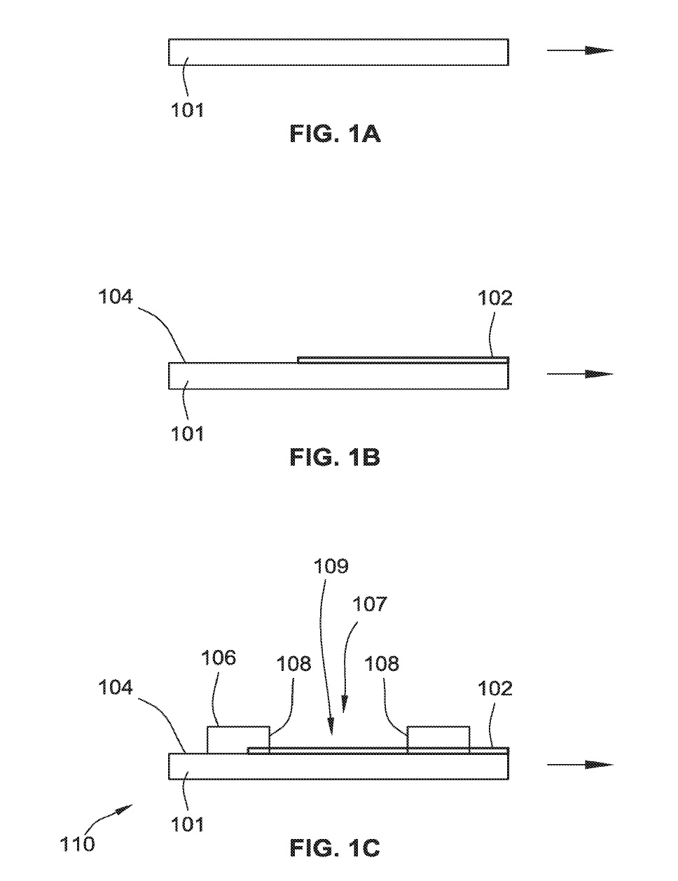

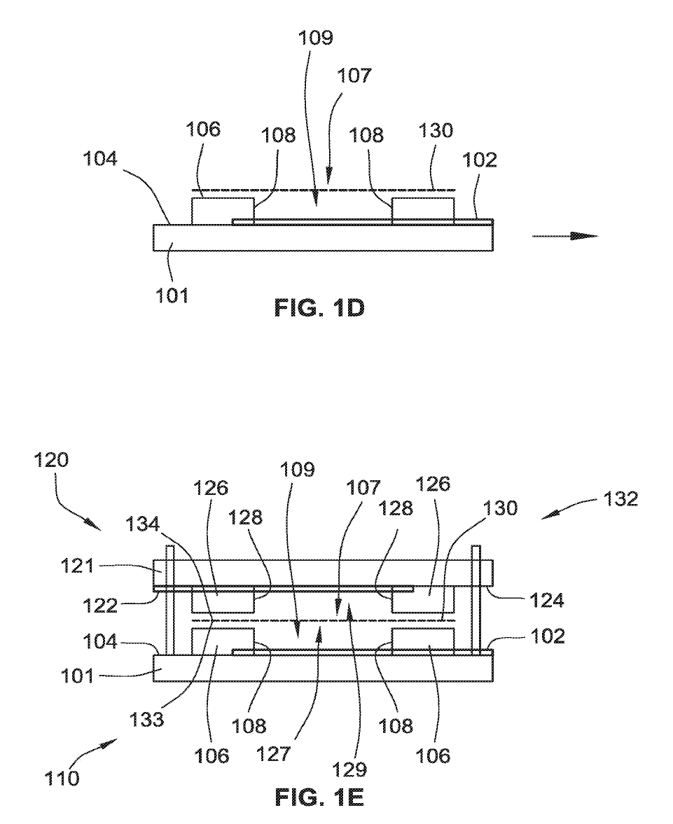

Referring to FIGS. 1A-1E, a fabrication process is directed to full integration of electrodes (carbon-based, semi-conductor, or metal) into organs on chip (“OCC”) device using polycarbonate base substrates. More details in reference to one or more features of the OOC device are described, for example, in U.S. Pat. No. 8,647,861, titled “Organ Mimic Device With Microchannels And Methods Of Use And Manufacturing Thereof,” issued on Feb. 11, 2014, and which is incorporated by reference in its entirety.

Initially, referring specifically to FIG. 1A, first material substrate 101 is provided, e.g., a substrate in the form of a polycarbonate (“PC”) 02 plasma. The first material substrate 101 is generally a transparent substrate that is first cleaned and plasma activated, and subsequently patterned with metal electrodes. In other embodiments, the substrate is not transparent, e.g., it is opaque. In accordance with some embodiments, the first material substrate 101 is a polymer, including polycarbonate, styrene-ethylene/butylene-styrene (“SEBS”), polydimethylsiloxane, polyurethane, polyester, cyclic olefin copolymer (“COC”), cyclic olefin polymer (“COP”), epoxy-based negative photoresist SU-8, polymethylmethacrylate (“PMMA”), polyvinyl chloride (“PVC”), polystyrene (“PS”), polyimide, and/or polyethylene terephthalate (“PET”). In accordance with other embodiments, the first material substrate 101 is glass, silicon, and/or silicon nitride. In accordance with yet other embodiments, the first material substrate 101 includes at least one material selected from a group consisting of a flexible material and/or a stretchable material.

In reference to FIG. 1B, a first metallic film layer 102 is formed on a portion of an upper surface 104 of the first material substrate 101. For ease of description, the film layer 102 is referred as a metallic layer. However, in other examples, the film layer 102 (as well as other metallic film layers described below) is an electrically conductive layer that is not necessarily a metallic layer. A simple route to electrode patterning includes metal deposition through a shadow mask that is in contact with a transparent substrate. Thus, by way of further example, the first metallic film layer 102 is formed using metal deposition through a shadow mask in contact with the first material substrate 101. By way of another example, the first metallic film layer 102 includes a first layer of titanium (“Ti”) having a thickness of about 3 nanometers (“nm”), a second layer of gold (“Au”) having a thickness of about 25 nm, and a third layer of Ti having a thickness of about 1 nm. In other embodiments, the first metallic film layer 102 has a thickness in the range of approximately 10-30 nm. In other embodiments, the layer 102 and/or layer 103 has a thickness greater than 30 nm. In yet other non-limiting examples, and depending on the material selection for the first material substrate 101, other microfabrication techniques include photolithography, metal lift-off, and laser ablation. The resulting patterned substrate 101 with the layer 102 is activated in an oxygen plasma, and immediately functionalized with amino-silane, such as (3-aminopropyl) triethoxysilane (APTES), to introduce both hydroxyl and amine groups at the substrate surface (e.g., the upper surface 104). In accordance with some embodiments, the first metallic film layer 102 includes at least one material selected from a group consisting of a flexible material and/or a stretchable material.

In reference to FIG. 1C, a first polymeric layer 106 is attached to the upper surface 104 of the first material substrate 101. The first polymeric layer 106 forms a first opened microchannel 107 having two sides 108 separated by a gap 109. The first opened microchannel 107 contains the first metallic film layer 102, which extends across the first opened microchannel 107. In one example, the first polymeric layer 106 is a thin layer of patterned polydimethoxysiloxane (“PDMS”), which has been previously modified with epoxy-silane and which is aligned with the first material substrate 101 bearing the first metallic film layer 102. The first layer of PDMS 106 is pressed firmly against the first material substrate 101 to form a first assembly 110 that is baked overnight during a curing process at approximately 60° C. In other words, the first assembly 110 is a first microchannel assembly 110 that is formed from the first metallic film layer 102, the first material substrate 101, and the first polymeric layer 106, which together define the dimensions of the first opened microfluidic channel 107. According to one example, “epoxy-silane” refers to 3-Glycidyloxypropyl trimethoxysilane. In accordance with some embodiments, the first polymeric layer 106 includes at least one material selected from a group consisting of a flexible material and/or a stretchable material.

A similar and/or symmetrical second assembly 120 is made in accordance with the process described above in reference to the first assembly 110. The second assembly 120 (illustrated in FIG. 1E) includes a second material substrate 121, a second metallic film layer 122 having an upper surface 124, and a second polymeric layer 126 forming a second opened microchannel 127 having two sides 128 separated by a gap 129. In other words, the second assembly 120 is a second microchannel assembly 120 that is formed from the second metallic film layer 122, the second material substrate 121, and the second polymeric layer 126, which together define the dimensions of the second opened microfluidic channel 127. In some embodiments, the second metallic film layer 122 is symmetrically integrated with respect to the first metallic film layer 102. Optionally, the second metallic film layer 122 is generally formed and is identical to the first metallic film layer 102.

In reference to FIG. 1D, the first and second substrates 101, 121 are again modified with epoxy-silane, and a polymeric membrane 130 is placed in-between the two epoxy-treated substrates 101, 121. The membrane 130, according to one example, is a polycarbonate material that has been previously functionalized with APTES. Optionally, APTES, GLYMO, and/or other materials are used to bond electrode layers. In accordance with some embodiments, the membrane 130 is a polymer, including polycarbonate, SEBS, polydimethylsiloxane, polyurethane, polyester, COC, COP, SU-8, PMMA, PVC, PS, and/or PET. In accordance with other embodiments, the membrane 130 is glass, silicon, and/or silicon nitride. The membrane 130 has a first side 133 facing the first assembly 110 and a second side 134 facing the second assembly 120. In some embodiments, cells are adhered to the first side 133 of the membrane 130.

In reference to FIG. 1E, the two assemblies 110, 120 form a final assembly 132 by attaching the first opened microchannel 106 (containing the first metallic film layer 102) to a bottom side of the membrane 130 and the second opened microchannel 126 (containing the second metallic film layer 122) to a top side of the membrane 130. The first metallic film layer 102 and the second metallic film layer 122 each constitute transparent electrodes that are positioned with the membrane 130 therebetween. The final assembly 132 is pressed firmly and baked during a curing process at approximately 60° C. overnight. External contacts are added to the final device formed by the final assembly 132, the external contacts allowing connections of the patterned electrodes (i.e., metallic film layers 102, 122) to a measuring equipment.

The electrodes 102, 122 are not an add-on or an extra module to the OOC device but form part of the OOC device. The electrodes 102, 122 are integrated in the top and/or bottom channels of the OOC device and/or into/onto the membrane 130 to enable different measuring principles through the various biological layers present in the OOC device. The fabrication process enables the integration of the electrodes 102, 122 in flexible chips, rigid chips, and/or stretchable chips. The fabrication approach is extendable to other materials such as PET, COC, or COP. In other examples, the electrodes 102, 122 are integrated on the membrane 130, such that, for example, the electrode 102 is a first transparent electrode 102 that is positioned on a first side of the membrane 130, and the electrode 122 is a second transparent electrode that is positioned on a second side of the membrane 130. Alternatively, the electrodes 102, 122 on the membrane 130 are non-transparent (e.g., opaque). Alternatively yet, the electrodes 102, 122 and/or the membrane 130 are stretchable.

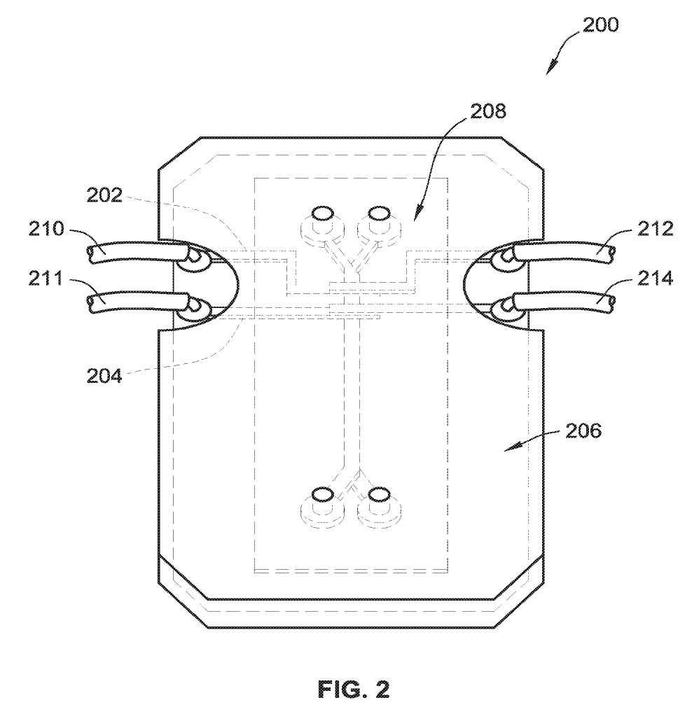

Referring to FIG. 2, an exemplary embodiment illustrates a device 200 fabricated using the process described above in reference to FIG. 1. The device 200 includes a pair of electrodes 202, 204 formed via respective metallic film layers in a body formed by transparent substrates 206. The electrodes 202, 204 are visible through the substrates 206 and are placed perpendicular to a microchannel 208. In alternative embodiments, the electrodes 202, 204 are placed parallel to the microchannel 208. In yet other alternative embodiments, the electrodes 202, 204 cover partially the microchannel 208, instead of covering the microchannel 208 entirely as illustrated in FIG. 2.

The electrodes 202, 204 are connected to electrical contacts 210-214 for coupling to a measuring device. The electrical contacts 210-214 are integrated into the substrates 206, with each of the electrical contacts 210-214 being electrically coupled with a respective end of the electrodes 202, 204. In other words, the electrical contacts 210-214 are electrically coupled to respective ends of the metallic film layers forming the respective electrodes 202, 204.

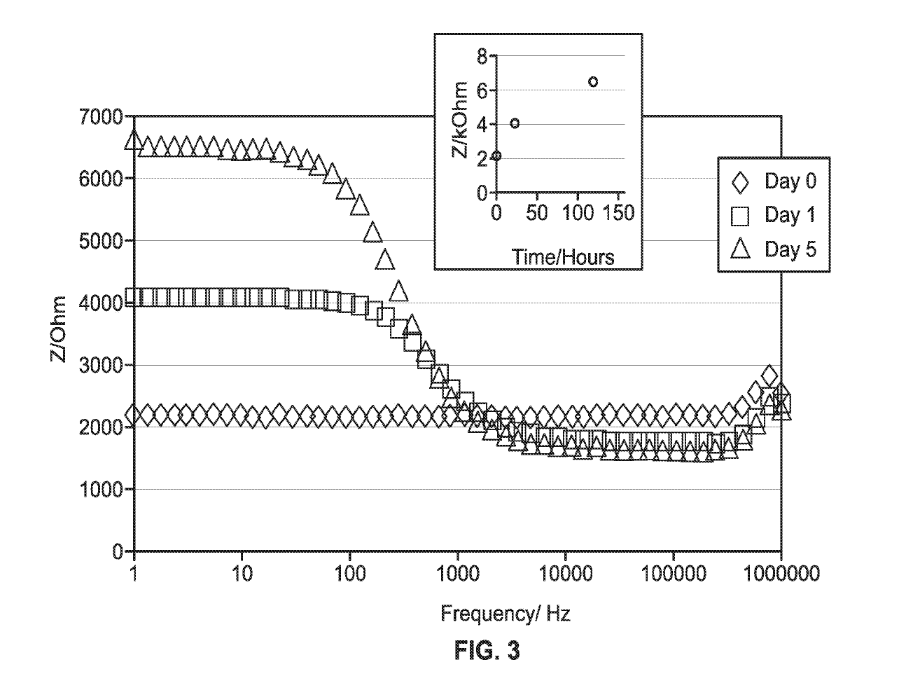

Referring to FIG. 3, a graph illustrates a four-electrode electrochemical impedance spectroscopy measured at different time point during the culture of Caco-2 cells.

Electrodes are Sufficiently Transparent to Allow Imaging

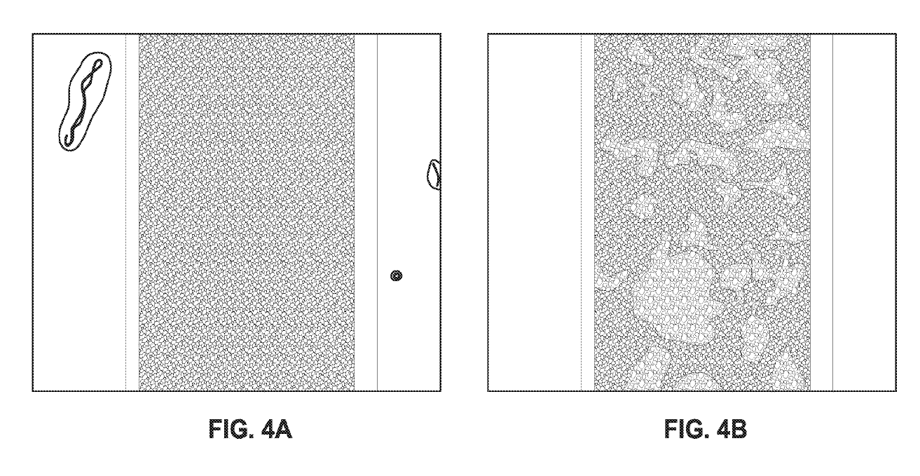

Referring to FIGS. 4A and 4B, low magnification phase contrast images of Caco-2 cells are cultured in a prototype TEER device, with the left image in FIG. 4A showing the Caco-2 cells in day one and the right image in FIG. 4B showing the Caco-2 cells in day five. According to some embodiments, ultra-thin film metal electrodes that are approximately 10-30 nm in thickness are preferred. Such thickness allows visualizing the cell culture through the electrodes. By way of example, a thin Ti/Au/Ti coating of 29 nm was used in the Caco-2 cells illustrated in FIGS. 4A and 4B to allow the imaging to pass through the electrode.

The Electrodes Allow Following Cell Growth, Differentiation and the Integrity of the Resulting Tissue

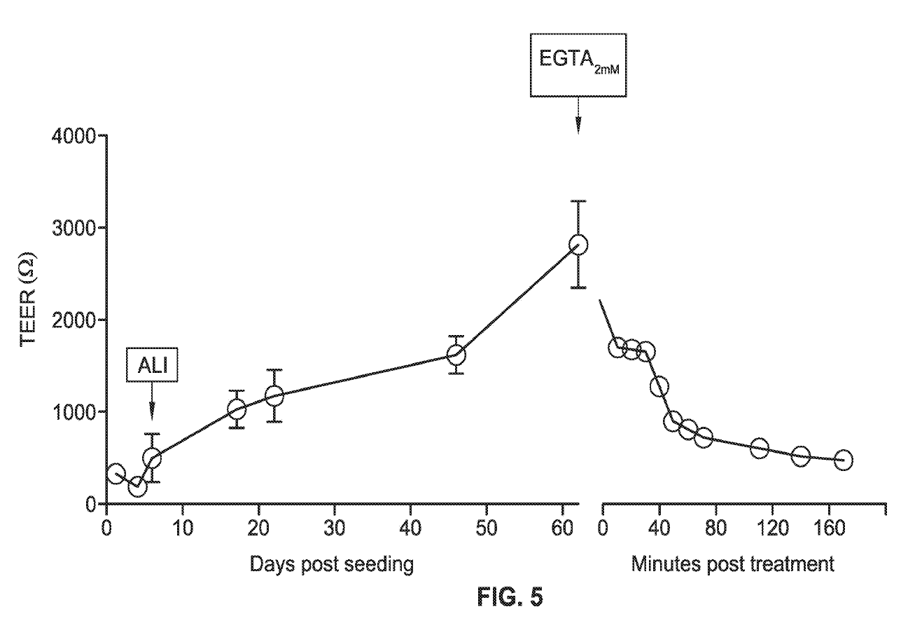

Referring to FIG. 5, a graph illustrates a time course experiment of ALI formation in a small-airway chip with integrated electrodes. ALI was formed for 70 days as seen by the increasing TEER value recorded by the electrodes and disrupted following the addition of ethylene glycol tetraacetic acid (“EGTA”) demonstrated by the rapid decrease in TEER signal.

Specifically, human primary airway epithelial cells were cultured and differentiated for 70 days using the TEER sensors integrated in 4 chips. TEER values were taken at different time points during differentiation process. Viability and quality of epithelium culture were assessed by light microscopy. Readouts included the following: epithelium morphology and integrity (no holes), cilia beating, and presence of mucus secretion. TEER was measured before and after the establishment of an air-liquid interface. TEER values were taken using a four-point impedance measurement method at 25 Hertz and data was presented as values ±scanning electron microscopy (“SEM”). EGTA 2 millimolar (“mM”) was used to disrupt tight junctions. An EGTA suspension was introduced in top and bottom channel sand measurements were taken every 10 minutes for 1 hour and then every 30 minutes.

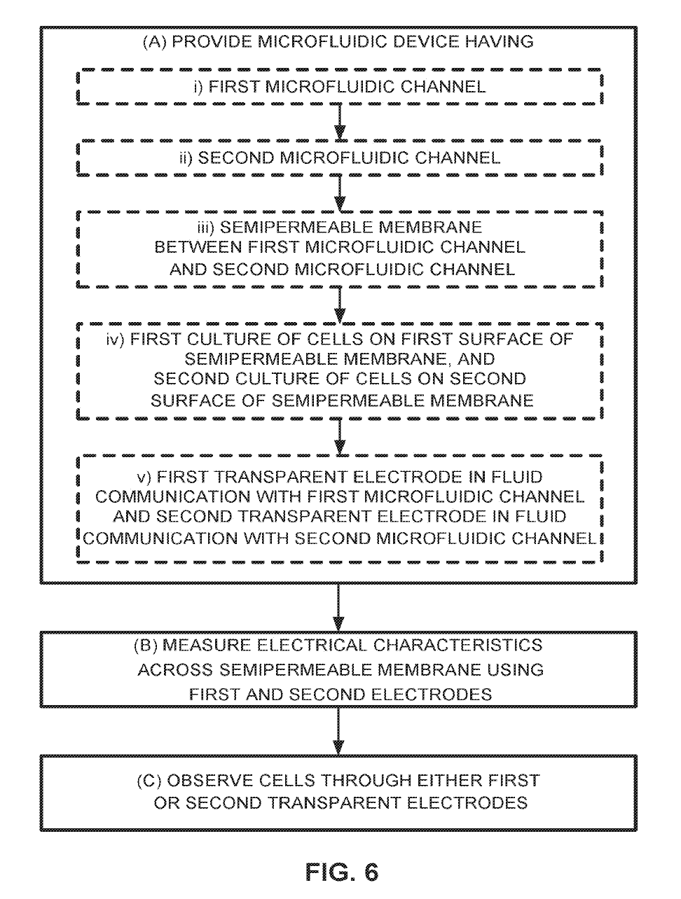

Referring to FIG. 6, a method is directed to measuring electrical characteristics across a membrane and includes (a) providing a microfluidic device that has (i) a first microfluidic channel, (ii) a second microfluidic channel, and (iii) a semipermeable membrane disposed between the first microfluidic channel and the second microfluidic channel. The semipermeable membrane includes first and second surfaces.

The microfluidic device further includes (iv) a first culture of cells on the first surface of the semipermeable membrane, and a second culture of cells on the second surface of the semipermeable membrane. The microfluidic device also includes (v) a first electrode in fluid communication with the first microfluidic channel and a second electrode in fluid communication with the second microfluidic channel, wherein the first and second electrodes are transparent.

The method further includes (b) measuring electrical characteristics across the semipermeable membrane using the first and second electrodes. Optionally, the method also includes (c) observing the cells through either the first or second transparent electrodes.

### APPLICATION EXAMPLES

The devices and methods described above refer, by way of example, to TEER as a main application. However, other applications may include at least one or more of the following:
measurement of other physiological parameters (e.g., short circuit current, and/or cell capacitance)
application of electric stimuli to cell cultures;
localized degradation of cell layer (e.g., electroporation, cell destruction), and induced wounds of controlled size and depth;
study healing, cell proliferation, cell migration across substrate and/or across membrane;
manipulation of cells (e.g., dielectrophoresis);
calibration of physical or mechanical stress applied to an OOC device (negative, pressure, positive pressure, torque, and/or shear stress);
measurement of flow rate in an OOC device;
generation and/or following of the formation or introduction of gas, and, more generally, bubbles; and/or
functionalization of electrodes (e.g., pH, ions, or oxygen sensor).

Other Features

According to one feature of the described devices and methods, a full integration of electrodes is achieved into a single OOC device. The full integration allows a sturdy set-up and stable measurements.

According to another feature, electrodes are integrated within different layers of the OOC (e.g., top, bottom, and/or membrane sides). This allows measuring through the various biological layers formed in the device.

According to yet another feature, the electrodes are flexible, stretchable, and sufficiently transparent. This permits imaging the cell culture through the electrodes to control the cell layer integrity.

According to yet another features, the electrodes are flexible, stretchable and non-transparent.

According to yet another feature, a fully integrated analytical solution is provided that is suited to the complexity of the OOC design to follow cell culture integrity, viability, and/or maturity over time. The approach allows the combination of transparent and semi-transparent electrodes within OOC. The electrodes are made of various flexible, stretchable, and/or rigid materials.

According to some exemplary embodiments, the electrodes include a material that is a metal, a semi-conductor, an oxide, a carbon, and/or a polymer. By way of example, the metal is gold, platinum, silver, and/or silver chloride. By way of another example, the semi-conductor is doped silicon. By way of further example, the oxide is indium tin oxide, titanium dioxide, and/or graphene oxide. By way of yet a further example, the carbon is graphite, fullerenes, and/or graphene. By way of yet another further example, the polymer includes conductive polymers having doped polyaniline, undopped polyaniline, polypyrrole, and/or polythiophene. By way of yet another further example, the polymer is conductive or semi-conductive via addition of conducting or semi-conducting species. According to an example, the conducting or semi-conducting species include nanoparticles and/or carboneous elements. By way of another example, the carboneous elements include carbon black, graphite, carbon nanotubes, fullerenes, graphene, and/or a combination thereof.

According to some other exemplary embodiments, the electrodes are coated with a conductive or insulating layer. In one example, the layer includes one or more polymers, organic mono-layers, organic polylayers, and/or oxides. In another example, the polymers include epoxy-based negative photoresist SU-8 and/or silicon nitride. In yet another example, the organic mono-layers or organic polylayers include a self-assembled monolayer of thiolated compounds or silane.

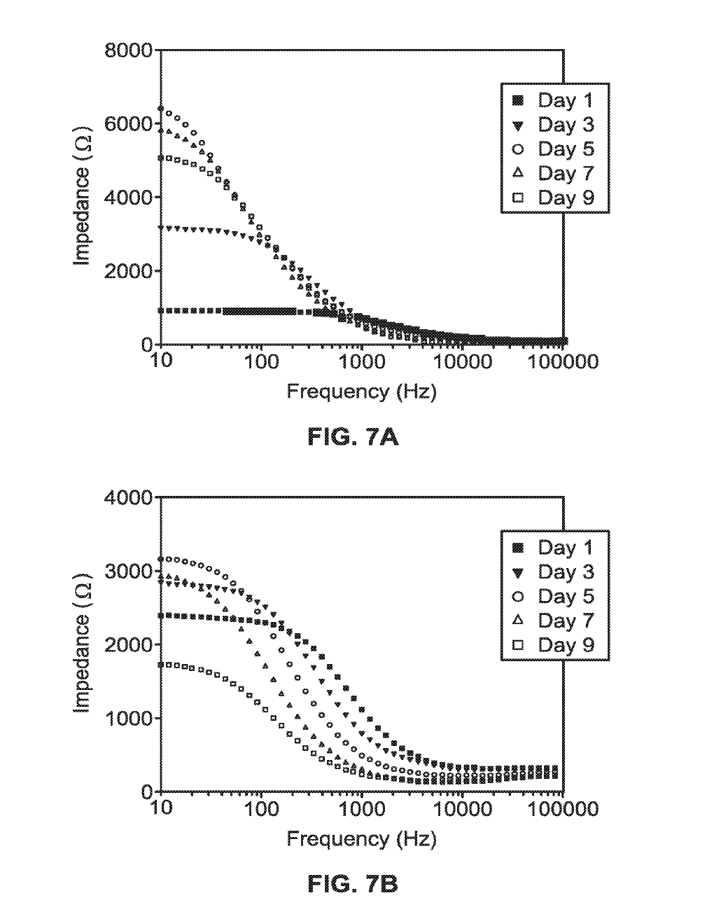

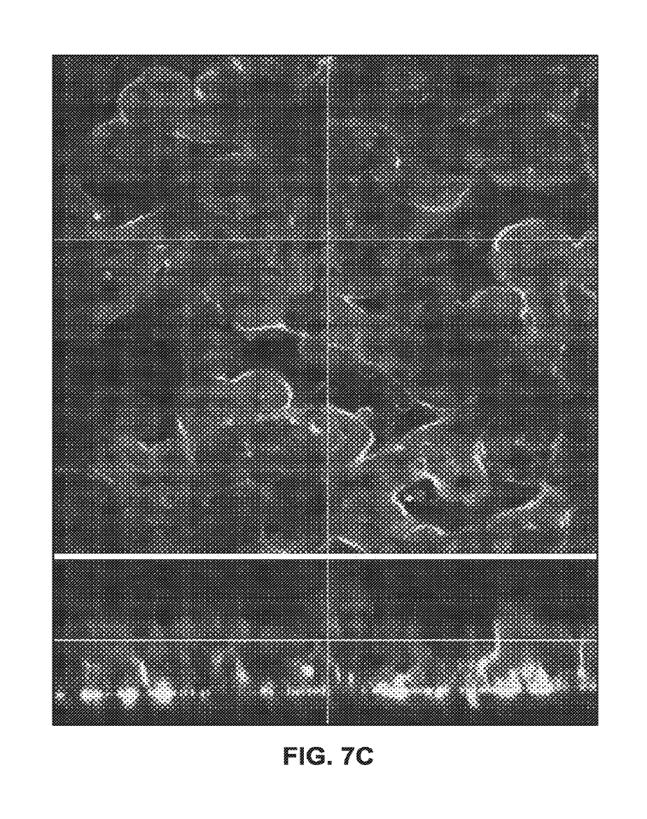

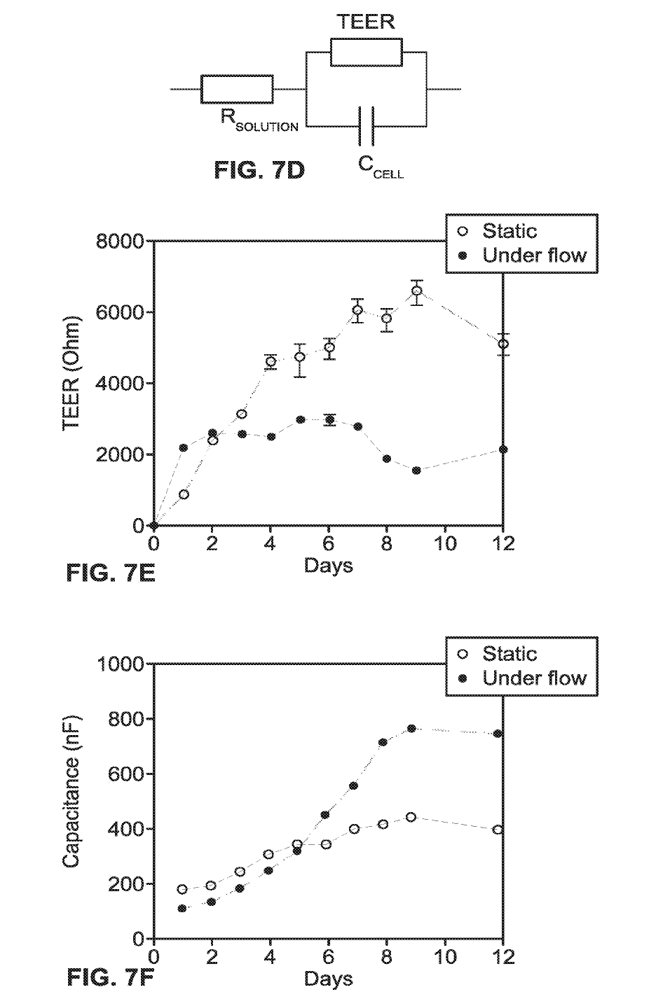

Referring to FIGS. 7A-7F, raw impedance spectra was recorded during the growth of Caco2 cells in OOC devices with integrated TEER sensors. CAco2 cells were grown under different conditions, with one type of conditions being static conditions in which media was replaced once a day, as illustrated by the data of FIG. 7A. Another type of conditions was under-flow conditions, in which the OOC devices were perfused continuously at a flow rate of 60 microliters (μL)/hour, as illustrated by the data of FIG. 7B.

More specifically, in accordance with another example of a measuring method, CAco2 cells were cultured under static and under flow condition. Under static conditions the culture media was refreshed once a day. Under flow conditions, a flow of culture media was continuously supplied at a flow rate of 1 μL/minute. Impedance measurements were taken once a day at varying frequencies.

The evolution of the impedance profiles changes considerably, depending on the culture conditions. Under the static conditions, curves overlay very well up to about 1,000 Hertz (“Hz”). The changes are measured at lower frequencies, reflecting the pure resistive nature of the tissue. Under the flow conditions, a strong capacitive component on the tissue rapidly develops and is observed as a variation in impedance at mid-frequency. The variation reflects the morphology of the tissue.

Under the static conditions, Caco2 cells do not form three-dimensional (“3D”) vili-like structures. Thus, the measurements did not perturbate the growth of the tissue as can be seen in FIG. 7C. However, under the flow conditions the vili-like structures form after day 6, which is a confocal fluorescence micrograph taken from the tissue located between electrodes showing healthy vili formation. The electric field applied during measurement did not damage the tissue located between the electrodes. The fluorescent confocal micrograph of FIG. 7C illustrates the Caco2 cell culture that was located between the electrodes after 9 days of culturing under flow conditions. As illustrated, the tissue is healthy and includes a 3D structure.

Referring specifically to FIG. 7D, an equivalent electric circuit was used to extract both TEER (i.e., barrier function) and Capacitance (i.e., surface area) values from the raw data presented in FIGS. 7A and 7B. Thus, the impedance response presented in FIGS. 7A and 7B were fitted to the electrical model presented in FIG. 7D to extract both TEER and CAPACITANCE values. Although the model in FIG. 7D is a simple representation of the electrical properties of the cell cultures, more complex models can be used to better fit to the measured data.

Referring specifically to FIGS. 7E and 7F, the evolution of the TEER and Capacitance values is depicted during the culture time under the respective, different conditions. While TEER expresses the para-cellular resistance, i.e., the junctions between cells (e.g. tight and/or adherens), Capacitance models the surface area of the cell culture. For example, the Capacitance values for cells cultured under static conditions increased slightly over the course of the experiment, while the Capacitance values for cells cultured under flow conditions increased steadily until day 7 and, then plateaued.

The measurements illustrated in FIGS. 7E and 7F reflect the evolution of the cell culture morphology over time. Caco2 cells cultured under static conditions grow as a flat layer. The TEER values increase, reflecting the increased tightness of the cell junctions, but the surface area of the culture remains approximately the same over time (as shown by the Capacitance). However, cells cultured under flow conditions rapidly grow to eventually form vili-like structures after day 7. The dip in TEER values also reflects this growth, as TEER is proportional to the tissue area. Capacitance values are potentially used to normalize TEER against absolute tissue area.

Thus, under static conditions, the cell culture remains flat (i.e. cell monolayer) and TEER increases steadily until day 10. This is reinforced by very little variation in Capacitance. Under flow conditions, TEER values rapidly increase and stabilize until day 6 after which the values decreases=to stabilize again at day 9. This decrease was seen to match the formation of 3 dimensional, vili-like structures as shown in FIG. 7C. Capacitance increased steadily until day 9 at which point it plateaued. Capacitance is therefore able to provide direct insight into the morphological changes of the tissue. TEER in conjunction with Capacitance measurements, therefore, offer a very complete picture of the growth and evolution of the tissue in real-time without needs for imaging.

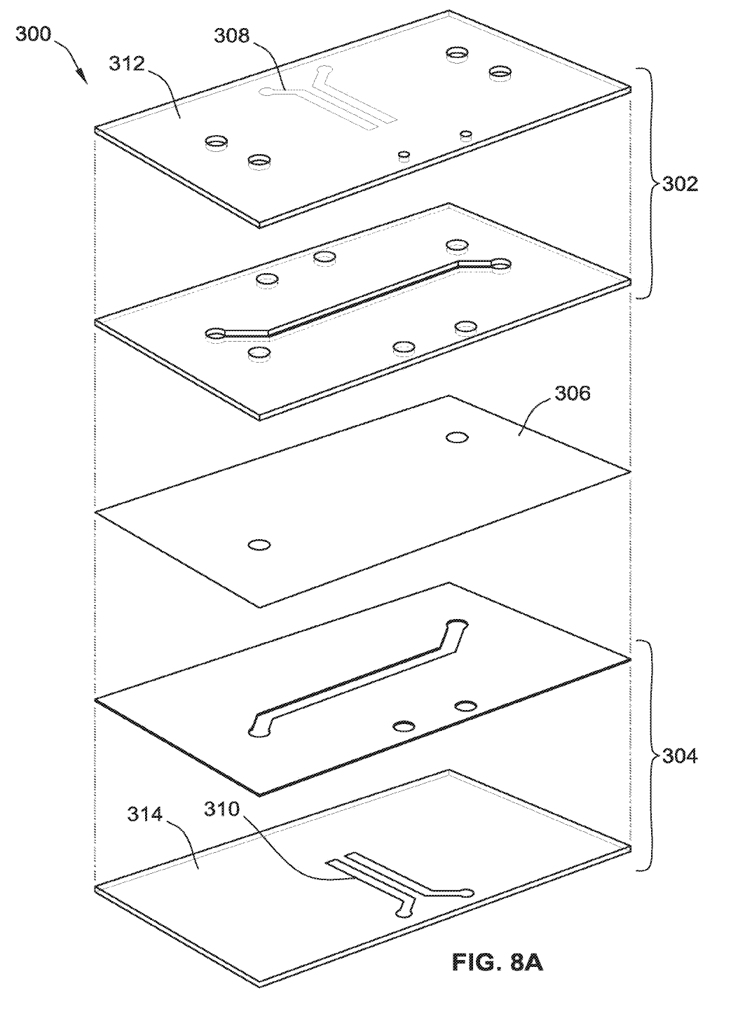

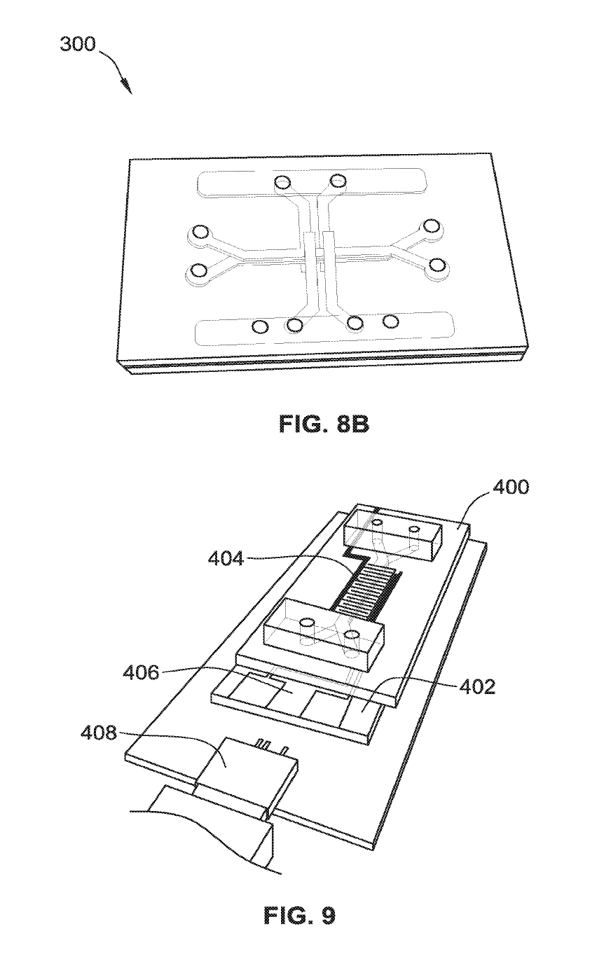

Referring to FIGS. 8a and 8B, an exemplary TEER chip 300 includes a top PDMS layer 302 having a thickness of about 1 mm, and a bottom PDMS layer 304 having a thickness of about 0.2 mm. The layers 302, 304 are separated by a PET membrane 306. Each layer 302, 304 has respective thin gold electrodes 308, 310 of about 25 nm on a respective polycarbonate substrate 312, 314. The layers 302, 304 define the microchannels of the TEER chip 300.

Referring to FIG. 9, according to another exemplary embodiment, an electric connection of OOC integrated electrodes to required external instrumentation is achieved through conventional connectors. Specifically, according to one example, an OOC device 400 is mounted onto a printed circuit board (“PCB”) 402 and electrodes 404 are connected to the PCB 402 using conductive ink or paste. A micro-USB connector 406 is soldered onto the PCB 402. A USB cable 408 is used to connect the OOC device 400 to the external instrumentation.

According to another exemplary embodiment, the connections 406, 408 are defined directly onto the OOC device 400 into a shape and size that allow connecting the electrodes 404 directly to external instrumentation without the need for a PCB or any other interfacing circuitry (e.g., flexible and/or stretchable printed electronic).

Optionally, in alternative embodiments, the connection to external instrumentation is a permanent connection or a temporary connection. Optionally, yet, additional electronic components are integrated onto the PCB 402 or directly into the OOC device 400. According to another optional aspect, connectors include spring loaded connectors, insertion connectors, flexible connectors, and/or connectors typically used in the electronic, microelectronic, and semi-conductor industries. According to yet another optional aspect, permanent or temporary conductive inks and paste, isotropic and anisotropic conductive tapes are used directly or in combination with connectors.

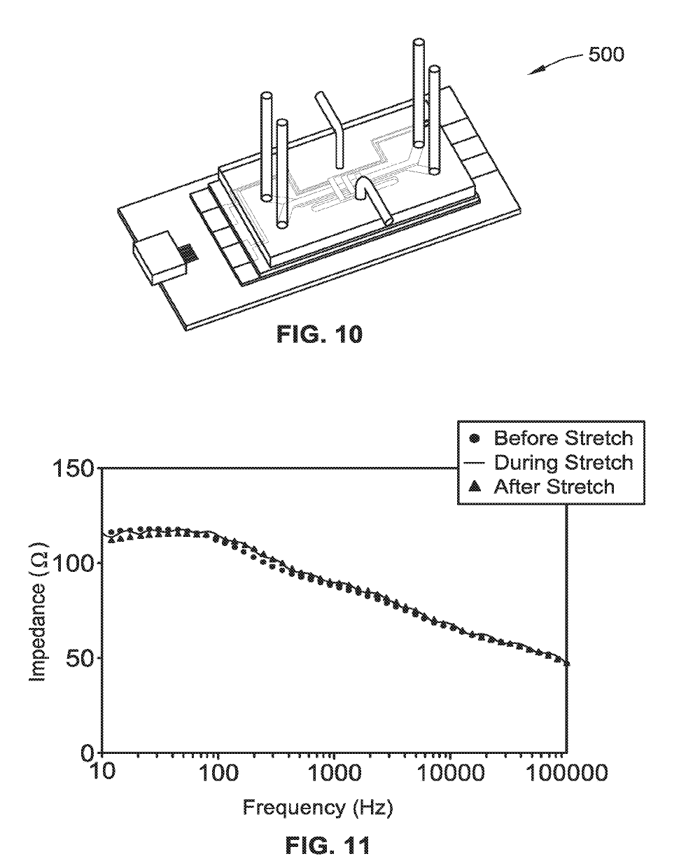

Referring to FIGS. 10 and 11, according to another exemplary embodiment, a device is directed to having electrodes that enable measurement of parameters in an OOC device 500 to which physical stress is applied. The physical stress, for example, includes (but is not limited to) stretching, strain, compression, torque, and/or shear stress. Physical and mechanical stress are optionally continuous or cyclic. Optionally, several forces are applied in combination or sequentially. The measurements, as specifically shown in FIG. 11, are taken during, before, and/or after the stress is applied. According to one alternative feature of the OOC device 500, a connector is on a separate substrate. According to another alternative feature of the OOC device 500, the connector is located directly on the chip.

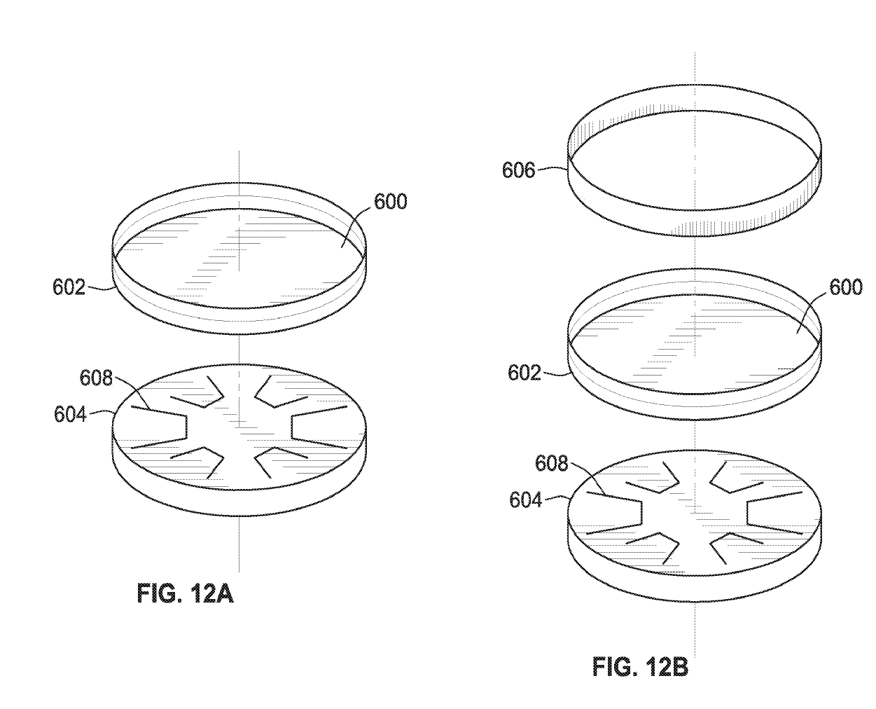

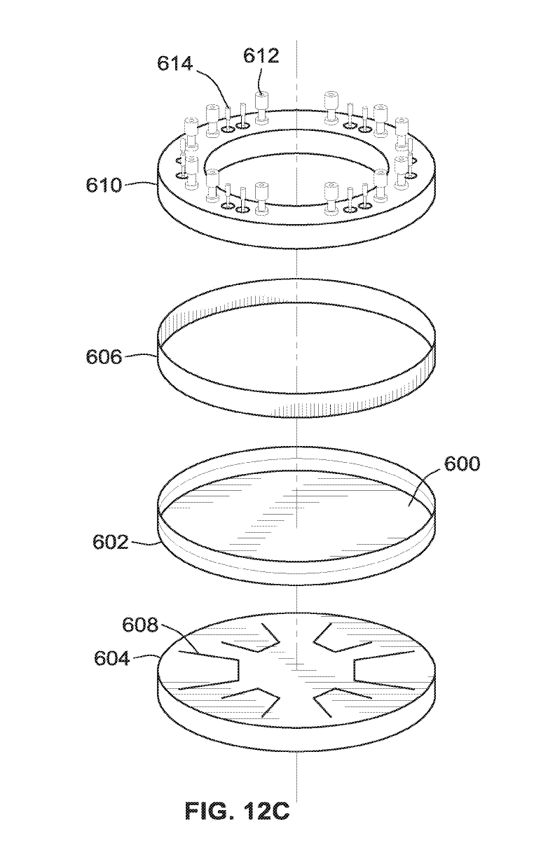

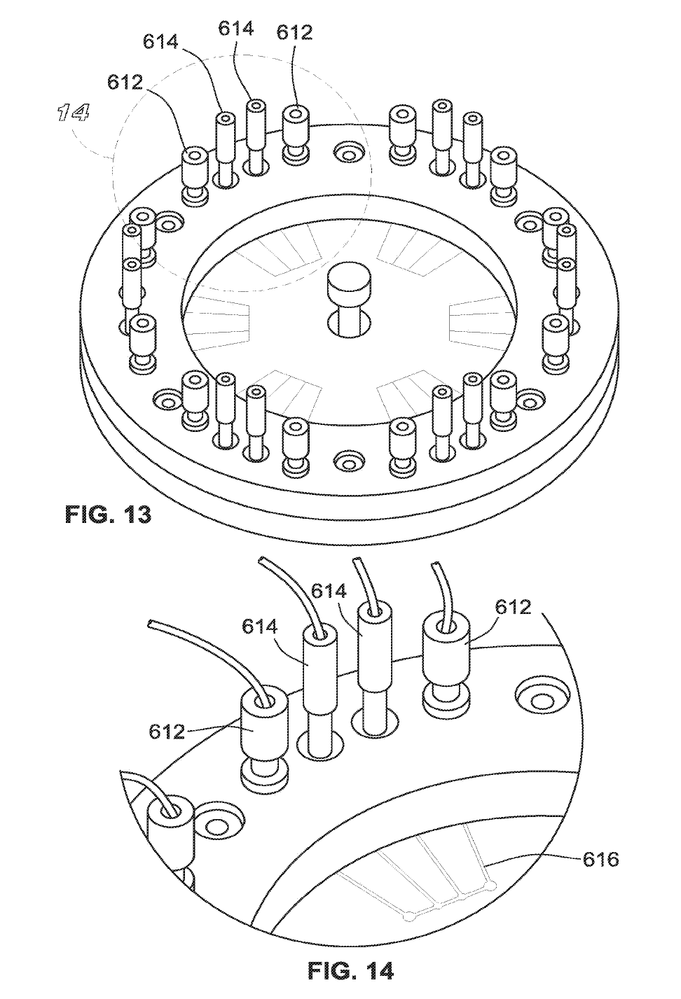

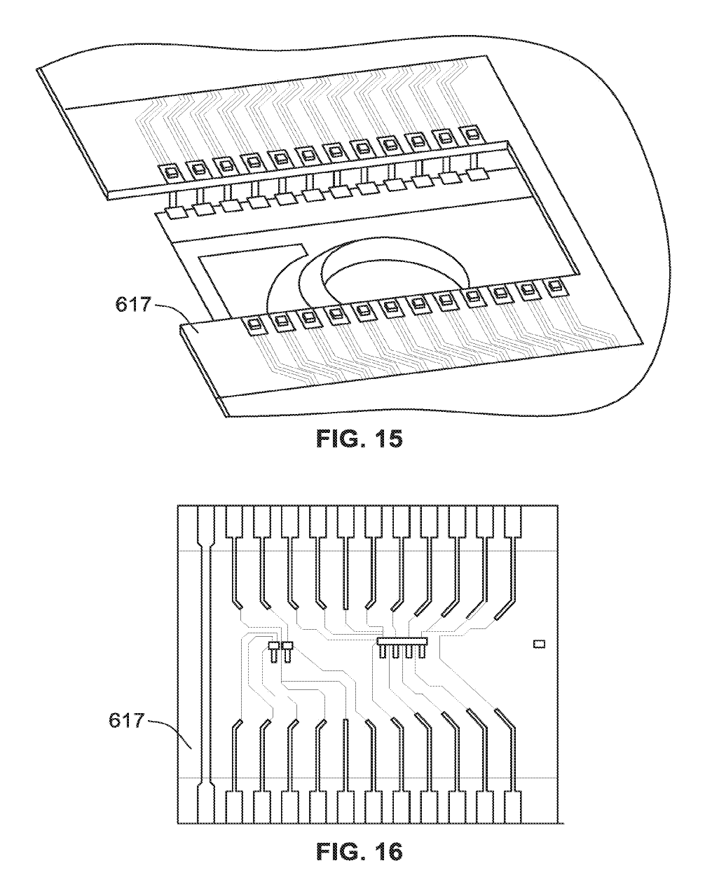

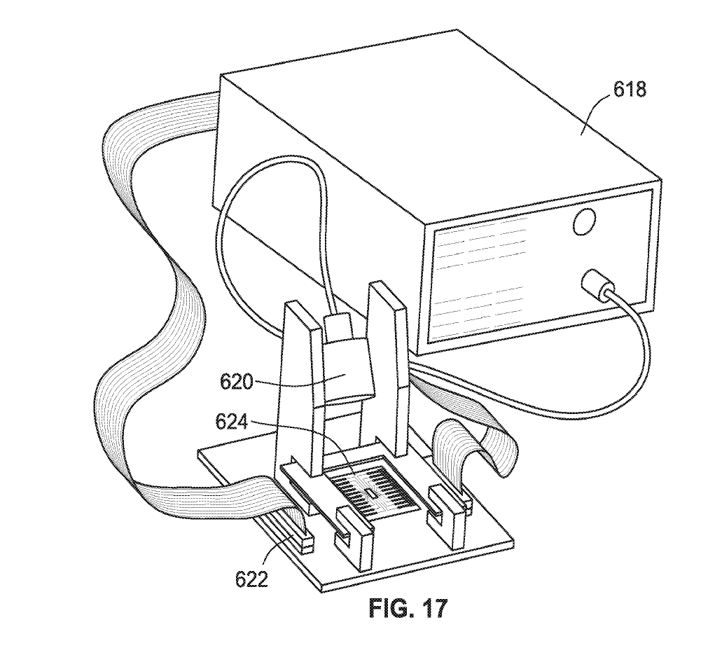

Referring generally to FIGS. 12A-17, an alternative embodiment includes reversibly contacting chip electrodes with pogo pins or similar features. Specifically, FIGS. 12A-14 illustrate a sealable interface with a top sealing plate 600, a PDMS gasket 602, a bottom compression plate 604, a top compression plate 606, a microfluidic device 608, a plexiglass ring 610, fluidic inlet and outlet connections 612, electrical inlet and outlet connections 614, and a microfluidic channel 616. FIGS. 15 and 16 show standard contacts on a printed circuit board 617 that enable customized automated actuation of devices. FIG. 17 shows an automated digital microfluidic (“DMF”) platform including a high-voltage amplifier 618, a webcam 620, a pogo-pin connector 622, and a DMF device 624.

Each of these embodiments and obvious variations thereof is contemplated as falling within the spirit and scope of the invention. Moreover, the present concepts expressly include any and all combinations and sub-combinations of the preceding elements and aspects.

1. A method for fabricating electrodes for a microchannel device having a membrane, the method comprising:
forming a first electrically conductive film layer on a portion of an upper surface of a first material substrate;
attaching a first polymeric layer defining the dimensions of the microfluidic channel to the upper surface of the first material substrate to form a first opened microchannel containing the first electrically conductive film layer, the first electrically conductive film layer extending across the first opened microchannel;
forming a second electrically conductive film layer on a portion of a lower surface of a second material substrate;
attaching a second polymeric layer defining the dimensions of the microfluidic channel to the lower surface of the second material substrate to form a second opened microchannel, the second electrically conductive film layer extending across the second opened microchannel; and
attaching the first opened microchannel containing the first electrically conductive film layer to a bottom side of the membrane and the second opened microchannel containing the second electrically conductive film layer to the top side of the membrane, the first electrically conductive film layer and the second electrically conductive film layer each constituting electrodes and being positioned with the membrane therebetween, wherein at least one of the electrodes is transparent to light.

2. The method of claim 1, wherein the first electrically conductive film layer, the first material substrate, and the first polymeric layer defining the dimensions of the microfluidic channel form a first microchannel assembly, the second electrically conductive film, the second material substrate, and the second polymeric layer defining the dimensions of the microfluidic channel form a second microchannel assembly, the first microchannel assembly and the second microchannel assembly being symmetrical.

3. The method of claim 1, further comprising integrating a plurality of electrical contacts into the first material substrate and the second material substrate and/or the membrane, each of the plurality of electrical contacts being electrically coupled with a respective end of the first electrically conductive film layer and the second electrically conductive film layer.

4. The method of claim 3, further comprising integrating a connection to the plurality of electrical contacts for enabling connecting the electrodes to external electronics and instrumentation.

5. The method of claim 1, wherein the material substrate is a polymer, including polycarbonate, styrene-ethylene/butylene-styrene (SEBS), polydimethylsiloxane, polyurethane, polyester, cyclic olefin copolymer (COC), cyclic olefin polymer (COP), SU-8, polymethylmethacrylate (PMMA), polyvinyl chloride (PVC), polystyrene (PS), and/or polyethylene terephthalate (PET).

6. The method of claim 1, wherein the material substrate is glass, silicon, and/or silicon nitride.

7. The method of claim 1, wherein the membrane is a polymer, including polycarbonate, styrene-ethylene/butylene-styrene (SEBS), polydimethylsiloxane, polyurethane, polyester, cyclic olefin copolymer (COC), cyclic olefin polymer (COP), silicon nitride, SU-8, polymethylmethacrylate (PMMA), polyvinyl chloride (PVC), polystyrene (PS), and/or polyethylene terephthalate (PET).

8-15. (canceled)

16. The method of claim 1, wherein at least one of the first electrically conductive film and the second electrically conductive film consists of a plurality of layers including one or more titanium layers and at least one gold layer.

17. The method of claim 16, wherein the plurality of layers includes a first titanium layer having a thickness of about 3 nanometers, a second gold layer having a thickness of about 25 nanometers, and a third titanium layer having a thickness of about 1 nanometers.

18. The method of claim 1, wherein the electrodes include a material selected from a group consisting of a metal, a semi-conductor, an oxide, a carbon, and a polymer.

19. The method of claim 18, wherein the metal includes a material selected from a group consisting of gold, platinum, silver, and silver chloride.

20-31. (canceled)

32. A method for fabricating electrodes for a microchannel device having a membrane, the method comprising:
forming a first electrically conductive film layer on a portion of an upper surface of a first material substrate;
attaching a first polymeric layer defining the dimensions of the microfluidic channel to the upper surface of the first material substrate to form a first opened microchannel containing the first electrically conductive film layer, the first electrically conductive film layer extending across the first opened microchannel;
forming a second electrically conductive film layer on a portion of a lower surface of a second material substrate;
attaching a second polymeric layer defining the dimensions of the microfluidic channel to the lower surface of the second material substrate to form a second opened microchannel, the second electrically conductive film layer extending across the second opened microchannel; and
attaching the first opened microchannel containing the first electrically conductive film layer to a bottom side of the membrane and the second opened microchannel containing the second electrically conductive film layer to the top side of the membrane, the first electrically conductive film layer and the second electrically conductive film layer each constituting electrodes and being positioned with the membrane therebetween, wherein at least one of the electrodes has a thickness such that it is transparent to light.

33-39. (canceled)

40. A device containing electrodes, the device comprising:
a body having a first microchannel and a second microchannel;
a membrane located at an interface region between the first microchannel and the second microchannel, the membrane including a first side facing toward the first microchannel and a second side facing toward the second microchannel, the first side having cells adhered thereto; and
a first electrode positioned on a first side of the membrane and a second electrode positioned on a second side of the membrane, wherein at least one of the electrodes is transparent to light.

41. The device of claim 40, wherein the first electrode is symmetrically integrated with respect to the second electrode.

42. The device of claim 40, further comprising electrical contacts directly integrated in one or more of the body and the membrane such that each is electrically coupled with a respective end of the first and second electrodes.

43. The device of claim 40, wherein at least one of the body, the membrane, and the electrodes includes at least one material selected from a group consisting of a flexible material and a stretchable material.

44. The device of claim 40, wherein at least one of the first electrode and the second electrode has a thickness in the range of approximately 10-30 nanometers.

45. The device of claim 40, wherein at least one of the first electrode and the second electrode consists of a plurality of layers including one or more titanium layers and at least one gold layer.

46. The device of claim 40, wherein at least one of the first electrode and the second electrode is transparent to light.

47. A device containing electrodes, the device comprising:
a body having a first microchannel and a second microchannel;
a membrane located at an interface region between the first microchannel and the second microchannel, the membrane including a first side facing toward the first microchannel and a second side facing toward the second microchannel, the first side having cells adhered thereto; and
a first electrode positioned on a first side of the membrane and a second electrode positioned on a second side of the membrane, wherein at least one of the first electrode and the second electrode has a thickness such that it is transparent to light.

48-51. (canceled)

52. A method of measuring electrical characteristics across a membrane, comprising:
(a) providing a microfluidic device having
i) a first microfluidic channel,
ii) a second microfluidic channel,
iii) a semipermeable membrane disposed between the first microfluidic channel and the second microfluidic channel, the semipermeable membrane comprising first and second surfaces,
iv) a first culture of cells on the first surface of the semipermeable membrane, and a second culture of cells on the second surface of the semipermeable membrane, and
v) a first electrode in fluid communication with the first microfluidic channel and a second electrode in fluid communication with the second microfluidic channel, wherein the first and second electrodes are transparent; and
(b) measuring electrical characteristics across the semipermeable membrane using the first and second electrodes.

53. The method of claim 52, further comprising (c) observing the cells through either the first or second transparent electrodes.

54. The method of claim 52, wherein the first and second electrodes include gold having a thickness such that it is transparent to light.

55. The method of claim 52, wherein the thickness of the gold is 25 nanometers or less.

56. The method of claim 52, wherein the first culture of cells includes epithelial cells and the measuring includes measuring transepithelial electric resistance (TEER).

57-64. (canceled)

65. A method of measuring electrical characteristics across a membrane, comprising:
a) providing a microfluidic device including
i) a first microfluidic channel,
ii) a second microfluidic channel,
iii) a semipermeable membrane disposed between the first microfluidic channel and the second microfluidic channel,
iv) a first culture of cells in the first microfluidic channel, and
v) electrodes in fluid communication with the first microfluidic channel; and
b) measuring electrical characteristics across the membrane by impedance spectroscopy.

66. The method of claim 65, wherein the membrane includes first and second surfaces, the first culture of cells being on the first surface of the semipermeable membrane.

67. The method of claim 66, wherein the microfluidic device further includes a second culture of cells on the second surface of the semipermeable membrane.

68. A device comprising: a membrane positioned between a top electrode and a bottom electrode, the top and bottom electrodes being connected to a detachable interface.

69. The device of claim 68, wherein the device is a microfluidic device and the membrane is positioned between first and second microchannels.

70. The device of claim 68, wherein the microfluidic device includes cells in the first or second microchannels, or both.

71-74. (canceled)
